(19) United States
(12) Patent Application Publication
(10) Pub. No.: US 2010/0183688 A1
(43) Pub. Date: Jul. 22, 2010

(54) Delivery System and Method of Manufacturing the Same

(75) Inventors: Limin Liu, Palatine, IL (US); Ralph Spindler, Palatine, IL (US); Stephen J. Urbanec, Arlington Heights, IL (US); Gholam-Reza Vakili-Tahami, Naperville, IL (US); Ashoke K. Sengupta, Barrington, IL (US); Kevin Cureton, Evanston, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: Amcol International Corporation, Hoffman Estates, IL (US)

(21) Appl. No.: 12/373,769

(22) PCT Filed: Jul. 23, 2007
(86) PCT No.: PCT/US07/16516

§ 371 Date: Dec. 18, 2009

Related U.S. Application Data
(60) Provisional application No. 60/832,880, filed on Jul. 24, 2006.

(51) Int. Cl. A61K 8/02; A61K 9/14; A01N 25/26; A61Q 15/00; A61Q 17/04; A61Q 11/00; A61K 31/60; A61K 33/00; A61K 31/23; A61K 31/355; A61K 31/341; A61K 31/327; A61K 31/555; A61K 31/12; A61K 31/222; A61K 31/19; A61Q 5/10

(52) U.S. Cl. 424/401; 424/484; 424/488; 424/485; 424/405; 424/59; 424/65; 424/49; 514/159; 424/600; 514/552; 514/458; 514/474; 514/714; 514/186; 514/686; 514/546; 514/557; 8/405

(57) ABSTRACT

A microparticle delivery system for an active compound which includes an active compound loaded onto polymeric microparticles, wherein the loaded microparticles are encased by a matrix material comprising about 68% to about 99%, by weight, of the microparticle delivery system. Compositions containing the microparticle delivery system, and methods of manufacturing the microparticle delivery system, also are disclosed.

### CROSS-REFERENCE TO RELATED APPLICATIONS

This application claims the benefit of U.S. provisional patent application Ser. No. 60/832,880, filed Jul. 24, 2006, incorporated herein by reference.

### FIELD OF THE INVENTION

The present invention relates to a composition and a method of stabilizing active or adjuvant compounds in a cosmetic, personal care, or pharmaceutical formulation, such that interactions between the active or adjuvant compound and a second active or adjuvant compound in the formulation, or with the formulation carrier, are eliminated or minimized. In one embodiment, the present invention relates to a composition and a method of enhancing the tanning rate of self-tanning compositions with a minimal adverse effect on the color of the composition during storage. More particularly, the present invention relates to a tanning composition containing (a) a self-tanning compound and (b) a self-tanning potentiator loaded onto (c) microparticles, wherein the loaded microparticles are encased in a matrix material, to provide a microparticle delivery system.

### BACKGROUND OF THE INVENTION

Stabilizing active compounds in a formulation is an important goal of researchers in the cosmetic, personal care, and pharmaceutical arts. Many active compounds are reactive, e.g., unstable, when present in a formulation, or, in some cases, are interactive with other actives or adjuvants that are present in a formulation. An improved stability of the active compound, and the formulation as a whole, is a particular goal of these researchers.

Examples of active compounds that may interact with other components in a formulation include retinoids, such as retinoic acid, retinol, retinaldehyde, and derivatives of these compounds. These retinoids are particularly sensitive to oxidation, reaction with other components in a formulation, and/or the formation of dimers or higher oligomers, which can be accelerated by other compounds in the formulation, such as acids, and in particular, alpha- and beta-hydroxyacids, such as lactic acid, glycolic acid, salicylic acid, and related compounds. Other examples of interactive active compounds include oil and water soluble vitamins, such as ascorbic acid and its derivatives, tocopherol and its derivatives, and vitamin K. Compounds such as benzoyl peroxide also can be stabilized to prevent interaction with other components in a formulation.

Many different approaches have been taken to improve active compound stability, and adjuvant compound stability, while maintaining the efficacy of these compounds. To date, no approach completely or sufficiently stabilizes these compounds.

One particular cosmetic formulation that is widely used by a relatively large portion of the population is a self-tanning composition, which darkens light colored skin through the use of a chemical-based tanning composition. Many individuals wish to avoid unnecessary exposure to ultraviolet solar radiation because of an increased risk of skin cancer. Therefore, alternative means of darkening the skin, i.e., self-tanning compositions, have increased in popularity.

One of the most widely used methods of enhancing a tan color is by a topical application of a self-tanning compound, such as dihydroxyacetone (DHA) in a suitable cosmetic formulation, to the skin. DHA forms a dimeric structure that converts to a monomeric form of DHA when contacted with water. Monomeric DHA darkens the skin through a reaction similar to the Maillard reaction by reacting with the free amino groups of skin proteins. Initially, the skin color formed after an application of DHA was unpredictable; and often was an orange hue rather than a desired brown color. By using more highly purified DHA, and improved formulations containing DHA, self-tanning compositions are more effective in producing the desired brown skin color.

One significant disadvantage of the DHA self-tanning approach is the length of time required (e.g., more than 4 and up to 12 hours) to observe a demonstrable darkening of the skin. Several different approaches have been attempted to improve the speed of the tanning process, including adding potentiators to tanning formulations. Typically, potentiators are primary or secondary amino-containing compounds. DHA reacts with a potentiator in a manner similar to the reaction with skin proteins to produce a rapid brown tan. A proper choice and formulation of a potentiator can provide a more natural tan color.

U.S. Pat. No. 5,603,923 discloses artificial tanning compositions comprising dihydroxyacetone and certain amino acids or their salts in a topical carrier at a pH less than 4. However, the compositions can lose about 20% tanning actives after three months storage at room temperature. This substantial loss of DHA is unacceptable from a product stability standpoint. U.S. Pat. No. 3,177,120 discloses the problem of including tanning actives, like DHA, with amino-group containing compounds in a single composition. A yellow or brown composition color developed during storage prior to topical application.

Although potentiators shorten the length of time to observe self-tanning results, tanning compositions containing a potentiator often are unstable with respect to color formation in the container. From a consumer acceptance perspective, this is a serious esthetic disadvantage. Furthermore, DHA that prematurely reacts with a potentiator is consumed and no longer available to tan the skin, and the effectiveness of the tanning composition therefore is reduced.

Several methods to overcome the problem of premature color formation in tanning compositions have been proposed, including first applying a potentiator solution to the skin, followed by an application of a DHA-containing formulation, or a vice versa application with a first application of DHA, then the potentiator (see, U.S. Pat. No. 5,503,874; U.S. Pat. No. 5,705,145; U.S. Pat. No. 5,705,145; and U.S. Pat. No. 6,399,048). Another approach utilizes a two-chamber package, wherein one chamber contains an emulsion incorporating a potentiator and the second chamber contains an emulsion incorporating DHA (see, U.S. Pat. Nos. 5,645,822 and 5,750,092). When applied to the skin, the contents of the two chambers mix such that the potentiator activates the DHA to enhance the rate of tanning. This approach is highly effective, but the cost of developing dual chamber packaging, and the cost to consumers, can be prohibitive. Therefore, a less costly method of obtaining the same result is highly desired.

WO 2005/030162 discloses a method of overcoming disadvantages associated with prior self-tanning compositions by loading a potentiator onto microparticles to provide a delivery system, then coating a wax or ester on the loaded delivery system. The coated and loaded delivery system is included in a self-tanning composition that contains DHA or other self-tanning compound, thereby preventing the potentiator from prematurely reacting with the DHA until the formulation is applied to the skin.

In order to improve self-tanning compositions, the present invention is directed to providing a single formulation that increases the tanning rate, while protecting the potentiator from prematurely reacting with the self-tanning compound until the tanning composition is applied to the skin. Premature darkening of the potentiated tanning composition therefore is avoided, which provides an extended shelf life for the product and improved customer efficacy and esthetics.

In one embodiment of the present invention, a self-tanning potentiator first is loaded onto microparticles, then the loaded microparticles are encased in a matrix material to protect the potentiator from prematurely reacting with self-tanning compounds, such as DHA, before topical application. After a formulation containing a present delivery system is applied to the skin; the potentiator is released and the self-tanning compound and potentiator react to promote the tanning rate. A formulation containing a delivery system of the present invention can be in the form of an oil-in-water emulsion, a water-in-oil emulsion, or a gel, for example, for topical application.

### SUMMARY OF THE INVENTION

The present invention is directed to delivery systems and formulations having an improved stability of an active compound or an adjuvant compound in a cosmetic, personal care, or pharmaceutical formulation, especially compositions that contain a second active or adjuvant compound that is interactive with the active or adjuvant compound. As used hereafter, the term “active compound” is synonymous to, and used interchangeably with, the phrase “active compound and/or adjuvant compound”.

One aspect of the present invention is to provide a stable formulation wherein an active compound is loaded onto a microparticles and the loaded microparticles are encased in a matrix material to provide a delivery system.

Still another aspect of the present invention is to provide a method of protecting an active compound loaded onto microparticles from interactions with a second active compound by encasing the loaded microparticles in a sufficient amount of a matrix material to avoid premature interactions or release of the active compound, i.e., prior to the application.

Yet another aspect of the present invention is to provide a composition comprising a water-soluble active compound, wherein the composition is in the form of an emulsion.

A further aspect of the present invention is to provide a composition comprising an oil-soluble active compound, wherein the composition is in the form of an emulsion.

Another aspect of the present invention is to provide a composition comprising an oil-soluble active compound, wherein the composition is based on a nonaqueous solvent, like an oil.

Another aspect of the present invention is to provide a composition containing an active compound selected from the group consisting of a skin care compound, a topical drug, an antioxidant, a dye, a self-tanning compound, an optical brightener, a deodorant, a fragrance, a sunscreen, a pesticide, a drug, and similar compounds, and mixtures thereof.

In a more detailed aspect, the present invention provides tanning compositions comprising a self-tanning compound and a protected self-tanning potentiator to enhance the rate of skin tanning.

In another detailed aspect, the present invention provides color-stable self-tanning compositions comprising (a) a self-tanning compound and (b) a self-tanning potentiator loaded onto polymeric microparticles, wherein said loaded microparticles are encased in a matrix material.

Yet another aspect of the present invention is to provide a method of protecting a potentiator loaded onto polymeric microparticles from interacting with a self-tanning compound in a composition by encasing the potentiator loaded polymeric microparticles in a matrix material.

These and other novel aspects of the present invention will become apparent from the following detailed description of the preferred embodiments.

### DETAILED DESCRIPTION OF THE INVENTION

A delivery system of the present invention comprises: (a) polymeric microparticles, (b) an active Compound, and (c) a matrix material. The matrix material comprises about 68% to about 99%, by weight, of the delivery system. The active compound can be water soluble or oil soluble.

As used herein, the term “microparticle” refers to a polymeric microparticle prior to loading of an active compound. The term “loaded microparticle” refers to a polymeric microparticle after loading with an active compound.

The matrix material is applied to the polymeric microparticles loaded with the active compound. The matrix material encases individual loaded microparticles and/or a plurality of loaded microparticles. If the active compound is water soluble, the matrix material preferably is hydrophobic. If the active compound is oil soluble, the matrix material preferably is hydrophilic. However, if the active compound is not appreciably soluble in the matrix material, any combination of active compound, hydrophilic or hydrophobic, can be used with the matrix material, hydrophilic or hydrophobic.

As used herein, the term “water-soluble compound” is defined as a compound having a solubility in water of at least 0.1 g (gram) per 100 grams of water at 25° C. Similarly, “oil-soluble compound” is defined as a compound having a solubility in mineral oil of at least 0.1 g per 100 grams of mineral oil, or similar nonaqueous solvent, at 25° C. The terms “water-dispersible” and “oil-dispersible” are defined as compounds having the ability to be suspended or dispersed in water or oil, respectively.

A delivery system of the present invention can be formulated with other ingredients to provide a semisolid or a liquid composition. The composition can be applied topically, such that the active compound is released from the delivery system after application to perform its intended function.

In one embodiment, the present formulations contain adsorbent polymeric microparticles loaded with a self-tanning potentiator. The loaded microparticles then are encased in a matrix material. In other embodiments, a different active compound is loaded onto the microparticles, followed by encasing by a matrix material.

In the self-tanning embodiment, potentiators that can be used to increase the rate of tanning, or the deepness of the tan, generally include amino-containing compounds. Self-tanning potentiators include the natural amino acids, like lysine, arginine, and glycine, and their salts, and compounds that contain amino groups, like diamines, triamines, and higher order amines, such as 1,2-ethanediamine, 1,3-propanediamine, 1,4-butanediamine, 1,6-hexamethylenediamine, diethylenetriamine, triethylenetetraamine, or derivatives or isomers of these amine compounds.

Other useful amine potentiators include, but are not limited to, N,N′-dimethylethylenediamine, N,N′-diethylethylenediamine, N,N′-diisopropylethylenediamine, N,N′-di-n-propylethylenediamine, N,N′-di-n-butylethylenediamine, N,N′-di-n-hexylethylenediamine, N,N′-dibenzylethylenediamine, N,N′-di-(2-carboxyethyl)-ethylenediamine, N,N′-di-(2-hydroxyethyl)-ethylenediamine, N-ethylethylenediamine, N-n-propylethylenediamine, N-isopropylethylenediamine, N-n-butylethylenediamine, N-secbutylethylenediamine, N-hexylethylenediamine, N-phenylethylenediamine, N-benzylethylenediamine, N-(2-hydroxyethyl)-ethylenediamine, N-(3-hydroxypropyl)-ethylenediamine, N-[3-trihydroxysilyl)-propyl]-ethylenediamine, N-[3-trihydroxysilyl)-propylkethylenediamine, N-[3-(trimethoxysilyl)-propyl]-ethylenediamine, and N-naphthylethylenediamine. Other diamines and derivatives of diamines are disclosed in U.S. Pat. Nos. 5,750,092 and 5,645,822, each incorporated herein by reference.

Polymeric amino-containing compounds useful as potentiators include, but are not limited to, siloxane polymers having pendant amino groups, such as those available from General Electric, Schenectady, N.Y. (e.g., GE SF 1706 or GE SF 1708) or Dow Corning Corp., Midland, Much. (e.g., DC 2-8566). Each of these amino-modified silicone polymers is known by the designated INCI name of amodimethicone. Methoxy amodimethicone/silesquioxane copolymer also can be used as a potentiator. Linear polyethylenimines, or branched versions of a similar polymer, also can be used as a potentiator, as can dendritic versions of amino polymers, such as those available from Dendritech, Inc. Midland, Mich., (PAMAM dendrimers) or from DSM, Galeen, Netherlands. Polyethyleneimines of the formula (CH₂CH₂NH)ₙ, wherein n ranges from 30 to 15,000, such as the EPOMIN™ products available from Aceto Corporation, Flushing, N.Y., and the POLYMIN™ products are available from BASF Corporation, Parsippany, N.J., also are potentiators. In addition, polymeric versions of amino acids, such as poly(lysine) and poly(arginine), can be used as a potentiator.

Adsorbent polymeric microparticles are widely used in personal care and pharmaceutical compositions. Such polymeric microparticles can have a high oil and a high water adsorbency, or a high oil or a high water adsorbency. The microparticles can be used to control the release rate of an active compound, to protect an active compound from decomposition, or to facilitate formulation of the active compound into a composition due to problems such as solubility or esthetics.

One class of adsorbent microparticles useful in the present invention is POLY-PORE® E200 (see U.S. Pat. Nos. 5,677,407; 5,712,358; 5,777,054; 5,830,967; and 5,834,577, each incorporated herein by reference). These microparticles, and related materials are commercially available from AMCOL International Corporation, Arlington Heights, Ill. Another class of adsorbent microparticles useful in the present invention is POLY-PORE® L200, as set forth in U.S. Pat. No. 5,830,960, incorporated herein by reference, also available from AMCOL International Corporation. Another adsorbent polymer is POLYTRAP®, also available from AMCOL International Corp, as disclosed in U.S. Pat. No. 4,962,170 and U.S. Pat. No. 4,962,133, each incorporated herein by reference.

Other adsorbent polymers that are commercially available include, for example, MICROSPONGE® (a copolymer of methyl methacrylate and ethylene glycol dimethylacrylate), available from AMCOL International Corporation, and Poly-HIPE polymers (e.g., a copolymer of 2-ethylhexyl acrylate, styrene, and divinylbenzene) available from Biopore Corporation, Mountain View, Calif.

The active compound, e.g., a potentiator, is incorporated, i.e., loaded, onto or into the adsorbent microparticles by spraying or adding the compound directly to the microparticles in a manner such that a homogeneous distribution of the compound on the microparticles is achieved. As used herein, the active compound is “loaded” onto the delivery system, i.e., is adsorbed, absorbed, and/or entrapped in the microparticles. Alternatively, the active compound first can be dissolved in a suitable solvent, then the resulting solution is sprayed or added to the microparticles. The solvent is removed by heating, vacuum, or both.

In one embodiment, the active compound, e.g. the amino-containing potentiator, first is loaded onto microparticles, followed by the application of a matrix material on the loaded microparticles, which modifies the release rate of the compound from the microparticles during storage and before a self-tanning formulation has been applied to the skin, and/or protects the potentiator loaded on the microparticles from prematurely reacting with self-tanning compounds in a formulation, such as DHA, during storage.

Thus, another aspect of the present invention is to provide a method of protecting an active compound from interacting with other ingredients in a formulation. In order to provide this benefit, microparticles loaded with a tanning potentiator are dispersed in a matrix material that encases the microparticles. These matrix materials are added, in their molten state, directly to the loaded microparticles in a manner such that a homogeneous distribution of the matrix material on the microparticles is achieved. Another method is to first disperse the microparticles loaded with the active compound in a matrix material, then regenerate microparticles through any of a number of methods known to those familiar with the art, followed by cooling the molten matrix material encasing the loaded microparticles to form solid microparticles. The resulting loaded microparticle/matrix particles can be further coated with a layer of a second matrix material that can be of a material identical to or different from the first matrix material, for example using a Wurster coater, in order to provide an added protective layer.

Stabilizing flavors or controlling drug release by coating a wax or polymeric material over an active compound has been widely used in pharmaceutical and food processing industries. Spray drying or spray congealing is a well-known technique of encapsulating active compounds in a solid matrix. U.S. Pat. No. 6,485,558, incorporated herein by reference, describes a spray-drying process for preparing organic pigment granules coated with a wax or polymer layer.

The spray congealing process is a solvent free and environmental friendly process. In a typical process, the active compounds and the carriers are admixed, then heated in a chamber to produce a molten mixture that is atomized into droplets. The droplets congeal to form microparticles.

Passerini et al., Journal of Controlled Release (2003), 88(2), 263-275, discussed using waxes in the preparation of microparticles with the ultrasonic spray congealing technique to control the in vitro release of verapamil HCl. By selecting the proper type and amount of carriers, microparticles with a spherical shape and good encapsulation efficiency were obtained. A zero-order release for 8 hours, without modifying the solid state properties of the drug, was observed. DE-A1-29 40 156 and WO 92/07912 disclose processes for producing wax-coated pigment powders using a fluidized bed process. In addition, WO 2005/053656 discloses a method of using an extruder to form a molten mixture of a labile drug and a carrier, then atomizing the molten mixture through an atomizer to produce multiparticulate drug particles. Such methods help reduce drug degradation. However, the surfaces of the active compounds, and especially hydrophilic active compounds, typically are incompletely coated by the wax. The control of the release rate of the active compounds also is limited.

Several of the adsorbent polymeric microparticles described above have both high oil and high water adsorbency. These microparticles have a unique capacity to be first loaded with a hydrophilic active compound, then the loaded microparticles can be dispersed in a hydrophobic matrix material. Alternatively, the adsorbent polymeric microparticle first can be loaded with a hydrophobic active compound, then dispersed in a hydrophilic matrix material.

A dispersion of loaded microparticles in either a hydrophilic or hydrophobic matrix material can be atomized into droplets by a number of well known methods. Several atomization methods can be used in the present invention, including (1) by pressure of single-fluid nozzles; (2) by two fluid nozzles; (3) by centrifugal or spinning-disk atomizers; (4) by ultrasonic nozzles; and (5) by mechanical vibrating nozzles. Detailed descriptions of atomization processes can be found in Lefebvre, “Atomization and Sprays” (1989) and in Perry's “Chemical Engineering Handbook” (7^{th} Ed. 1997). Optionally, the loaded microparticle/matrix particles can be further coated with a layer of a second matrix material through Wurster coater or similar fluidized bed coating technology. The second matrix material can be identical to or different from the first matrix material. In the Wurster technology, a coating solution is sprayed onto the fluidized particles, then the coating is allowed to dry, if a solvent is used, or to cool, if the second matrix material is in a molten state.

Preferably, a matrix material is hydrophobic when the active compound is water soluble. Conversely, the matrix material preferably is hydrophilic when the active compound is oil soluble. The preferred combinations of active compound and matrix material are not essential to the present invention because utilizing a hydrophilic matrix material with a water-soluble active agent, or a hydrophobic matrix material with an oil-soluble active compound, improves the properties of the composition.

The matrix material coats and encases the loaded microparticles. The matrix material, therefore, retards or eliminates a rapid displacement of the active compound from the loaded microparticles by water or a nonaqueous solvent.

The identity of the matrix material is not particularly limited. However, it is preferred that the matrix material is water insoluble, i.e., has a water solubility of 0.1 g (gram) or less in 100 ml (milliliter) of water at 25° C., when the active compound is water soluble. It is also preferred that the matrix material is oil insoluble, i.e., has an oil solubility of 0.1 g or less in 100 ml of mineral oil at 25° C., when the active compound is oil soluble. However, matrix materials having oil or water solubility up to 20 g in 100 ml of mineral oil or water, respectively, can be used with water-soluble and oil-soluble active compounds, respectively.

The matrix material is selected such that it does not adversely affect the active compound, e.g., is nonreactive and noninteractive with the active compound. The matrix material typically is a solid at room temperature, i.e., 25° C. In some embodiments, the matrix material has cosmetic or medicinal properties which perform in conjunction with the active compound.

Examples of suitable matrix materials are low melting (C8 through C20) alcohols and fatty alcohols ethoxylated with one to three moles of ethylene oxide. Examples of fatty alcohols and ethoxylated fatty alcohols include, but are not limited to, behenyl alcohol, caprylic alcohol, cetyl alcohol, cetearyl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Additional fatty alcohols and ethoxylated alcohols are listed in the “International Cosmetic Ingredient Dictionary and Handbook, Tenth Edition, volume 3” (2004), pages 2127 and pages 2067-2073, incorporated herein by reference. Another class of modifying compounds are the C8 to C20 fatty acids, including, but not limited to, stearic acid, capric acid, behenic acid, caprylic acid, lauric acid, myristic acid, tallow acid, oleic acid, palmitic acid, isostearic acid, and additional fatty acids listed in the “International Cosmetic Ingredient Dictionary and Handbook, Tenth Edition, volume 3” (2004), pages 2126-2127, incorporated herein by reference.

The matrix material also can be a hydrocarbon, like polydecene, paraffin, petrolatum, vegetable-derived petrolatum, or isoparaffin. Another class of matrix materials is waxes, like mink wax, carnauba wax, and candelilla wax, for example, and synthetic waxes, such as silicone waxes, polyethylene, and polypropylene. Fats and oils also can be useful modifying compounds which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the “International Cosmetic Ingredient Dictionary and Handbook, Tenth Edition, volume 3” (2004), pages 2124-2126. Other useful matrix materials are water-insoluble esters having at least 10 carbon atoms, and preferably 10 to about 32 carbon atoms. Numerous esters are listed in “International Cosmetic Ingredient Dictionary and Handbook, Tenth Edition, volume 3” (2004), pages 2115-2123.

Hydrophilic matrix materials can also be employed, including polyethylene glycols, polyethylene oxides, polyvinylalcohols, or cellulose based materials.

Self-tanning compositions of the present invention can be prepared in a variety of formulation types, including oil in water emulsion (o/w), water in oil emulsion (w/o), water in silicone emulsion (w/Si), anhydrous sticks, and aqueous gels. A loaded microparticle/matrix delivery system of the present invention can be incorporated into any of these formulation types. For example, an o/w emulsion can be prepared, and then microparticles, loaded with a potentiator and encased by a matrix material, can be added to the emulsion, preferably at the time preservatives and/or fragrances are added to the emulsion. Sufficient agitation is supplied to the emulsion to ensure that the loaded microparticle/matrix delivery system is homogeneously mixed into the composition. A similar method can be used to prepare other product types.

A tanning composition of the present invention contains a self-tanning compound in a sufficient amount to achieve a desired degree of tanning. The amount of self-tanning compound in the composition is well known to persons skilled in the art, but typically is about 0.1% to about 10%, preferably about 1% to about 7.5%, and more preferably about 1% to about 5%, by weight of the composition.

The amount of tanning potentiator included in the composition is sufficient to enhance the rate of tanning over a composition containing the same self-tanning compound, in the same amount, but absent a potentiator. Typically, a potentiator is present in the tanning composition in an amount of about 0.01% to about 10%, preferably about 0.1% to 5%, and more preferably about 0.1% to 2%, by weight of the composition.

The potentiator is incorporated into the tanning composition after loading onto polymeric microparticles and encasing of the loaded microparticles. The amount of microparticles in the composition is related to the desired amount of potentiator in the composition, and the amount of potentiator loaded onto the microparticles. Typically, the potentiator is loaded onto polymeric microparticles in an amount such that the loaded microspheres contain about 2% to about 80%, preferably about 5% to about 70%, and more preferably about 5% to about 50%, by weight, of the potentiator.

The weight percent of the matrix material in the loaded microparticle/matrix delivery system is about 68% to about 99%, preferably about 82% to about 95%, and more preferably about 84% to about 93%, by weight of the delivery system. More particularly, the loaded microparticle/matrix delivery system contains about 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, by weight, of a matrix material.

The release mechanism of the potentiator from the loaded microparticle/matrix delivery system onto the skin either can be from diffusion of the potentiator from the delivery system or a release of the potentiator through physical attrition of the loaded microparticle/matrix delivery system by the action of applying the tanning composition to the skin. These mechanisms allow the potentiator to form a film on the skin for reacting with the DHA, L-erytherulose, or other self-tanning compound in the composition.

An in vitro technique described by R. Jermann et al., Internatinal Journal of Cosmetic Chemistry ((2002), 24, 1-8), measures the rate of tan development. In this method, VITRO-SKIN™ (IMS, Milford, Conn.) is used as a substrate because its surface topography effectively mimics human skin, and it has lipid and protein components similar to human skin because it reacts with DHA to form a brown color. Color development can be recorded as a function of time by using a color meter (X-Rite, SP60). The color meter measures the L*, a*, and b* color parameters which can be compared to the same values for the original VITRO-SKIN substrate using the following equation:

ΔE(t)=((L*(0)−L*(0)²+(a*(0)−a*(t))²+(a*(0)−a*(0)²)^{1/2},

wherein L*(0) is the brightness value at time 0 before the tanning composition has been applied to the substrate and L*(t) is the brightness value at a time t after application of the composition, with similar values for a* and b* as a function of time. The rate of tanning, as measured by ΔE as a function of time, was found to increase more rapidly for compositions that included the potentiator compared to a control formulation, and in other cases, the final skin color also was darker as measured by the ΔE values.

The impact of adding a potentiator to a tanning composition on the color of the composition also was measured using a color meter. In comparison to the same amount of amodimethicone or lysine added directly to a composition, a composition containing potentiator-loaded microspheres exhibited a significant improvement in the color using either the ΔE or the Δb* index, such that, in some cases, the tanning composition had only a slight yellow color.

As demonstrated below, the present compositions are color stable because the potentiator is loaded onto the polymeric microspheres, which then are encased by matrix material, e.g., a wax or wax mixtures. In particular, the present compositions, when compared to an identical composition absent the loaded microsphere/matrix delivery system have a ΔE of about 6 or less after 12 weeks aging at 40° C.

An in vivo determination of self-tanning was performed by blocking out a defined area of skin, measuring skin color in that area with a color meter, and then applying a measured amount of the test formulation to the defined area. The color meter was used to record the skin color as a function of time after application of the test formulation.

### Examples

### Example 1

### Loading SF 1708 into POLY-PORE® E200

GE SF 1708, a silicone fluid available from General Electric Co. and having pendant amino groups (INCI name: amodimethicone) was loaded onto POLY-PORE® E200 by first dispersing the silicone fluid in a suitable solvent, e.g., heptane, then adding the resulting silicone dispersion in droplets to POLY-PORE® 200 under stirring stepwise. The silicone dispersion (50 g) (50% by weight SF 1708 in heptane) was loaded onto 50 g POLY-PORE® E200 microparticles and dried in a vacuum oven at 60° C. overnight. A free flowing powder was obtained wherein the weight percentage of SF 1708 was 33.3%.

### Example 2

### Loading SF 1708 onto POLYTRAP® 6603

Amodimethicone (50 g) was dispersed in 50 g heptane, then the 100 g of the resulting silicone dispersion was loaded into 50 g POLYTRAP® 6603 with stirring until the mixture became homogeneous. The amodimethicone-loaded microparticles were dried in a vacuum oven at 60° C. overnight. A free flowing powder was obtained wherein the weight percentage of SF 1708 was 50%.

### Example 3

### Loading Lysine onto POLY-PORE® E200

A lysine solution was prepared by dissolving 40 g of lysine in 40 g of DI (deionized) water. The mixture was stirred until the lysine was completely dissolved. The resulting aqueous lysine solution (30 g) was added to 90 g POLY-PORE® E200 microparticles with stirring in stepwise fashion. After mixing until homogeneous, the microparticles loaded with lysine were placed in a 60° C. vacuum oven overnight to remove the water. A free-flowing powder that contained 14.3% lysine, by weight, was obtained.

### Example 4

### Loading Lysine Hydrochloride into POLY-PORE® E200

A lysine hydrochloride solution was prepared by dissolving 20 g of lysine hydrochloride in 40 g of DI water. The mixture was stirred until the lysine hydrochloride was completely dissolved. The lysine hydrochloride aqueous solution (60 g) was added to 120 g POLY-PORE® E200 microparticles with stirring. After mixing until homogeneous, the microparticles were placed in a 60° C. vacuum over overnight to remove the water. A free-flowing powder that contained 14% lysine hydrochloride, by weight was obtained.

### Example 5

### Lysine Hydrochloride Loaded onto MICROSPONGE® 5640 in Two Steps

Lysine hydrochloride (50 g) was dissolved in 150 g water. The mixture was stirred until clear. The lysine hydrochloride solution (80 g) was added to 180 g MICROSPONGE® 5640 microparticles stepwise with stirring. Stirring was continued until the mixture was homogeneous. The loaded particles were placed in a 60° C. vacuum oven to remove the water. A free-flowing powder was obtained. Another 70 g of lysine hydrochloride solution (25% by weight) was added stepwise to 140 g obtained from the first loading step. This solution was added stepwise, and the resulting mixture was stirred until homogeneous. The particles were dried in an oven for 24 hours at 60° C. A free-flowing powder that contained 20% lysine hydrochloride, by weight, was obtained.

### Example 6

### Loading Lysine Hydrochloride into POLY-PORE® E100 in Three Steps

One hundred grams of lysine hydrochloride was added to 300 g DI water. The mixture was stirred until clear. The lysine hydrochloride solution (80 g) was added to 180 g POLY-PORE® E100 microparticles stepwise with stirring. Stirring was continued until the mixture became homogeneous. The loaded particles were placed in a 60° C. vacuum oven overnight to remove water. A free-flowing powder was obtained. For the next loading step, the 25% lysine hydrochloride solution (70 g) was added stepwise to 140 g of the product from the first loading step. The resulting mixture was stirred until homogeneous. The particles were dried in a vacuum oven for 24 hours at 60° C. A free flowing powder was obtained. Then, a third loading of 80 g of a 25% lysine hydrochloride solution was added to 140 g of the microparticles obtained from the second loading step and dried as described above. A free-flowing powder which contained 30 weight % lysine hydrochloride was obtained.

### Example 7

### Loading Lysine Hydrochloride into POLY-PORE® E100 in Four Steps

A loading solution was prepared by dissolving 120 g lysine hydrochloride in a mixture of 360 g water and 80 g acetone. The mixture was stirred until clear. For the first loading step, lysine hydrochloride solution (140 g) was added stepwise to 180 g POLY-PORE® E100 microparticles with stirring. Stirring was continued until the mixture became homogeneous. The loaded particles were placed in a 60° C. vacuum oven to remove the water. A free-flowing powder was obtained. Then, a second 140 g of the loading solution was added stepwise into the product obtained in the first loading. The solution was added in stepwise manner and the mixture was stirred until homogeneous. The particles were dried in a vacuum oven for 24 hours at 60° C. A free flowing powder was obtained. Then, a third 140 g solution was added and processed as described above. A free flowing powder was obtained. Finally, a fourth 140 g solution was added and stirred into the lysine hydrochloride loaded POLY-PORE® E100 particles until the mixture was homogeneous. The particles were dried again in a 60° C. vacuum oven. A free-flowing powder which contained 40% lysine hydrochloride, by weight, was obtained.

### Example 8

### Loading HYDROSIL™ 2776 onto POLY-PORE® E100 in Five Steps

HYDROSIL™ 2776, an alkoxysilane, also known as a silanol-substituted ethylenediamine and available from Degussa, USA, was loaded onto POLY-PORE® E100. For the first loading step, 80 g of the HYDROSIL™ aqueous solution (10%) was added to 160 g POLY-PORE® E100 microparticles stepwise with stirring. Stirring was continued until the mixture became homogeneous. The loaded particles were placed in a 60° C. vacuum oven overnight to remove the water. A free-flowing powder was obtained. Then a second 80 g portion of the HYDROSIL™ solution was added stepwise into the above obtained loading. Again, the solution was added stepwise and the mixture was stirred until homogeneous. The particles were dried in a vacuum oven for 24 hours at 60° C. A free flowing powder was obtained. Then, a third 80 g solution was added and dried as previously described. A free flowing powder was obtained. A fourth 80 g solution was added and stirred into the previously obtained loading until a homogeneous mixture was obtained. The loading was dried in a 60° C. vacuum oven. Finally, a fifth 80 g portion of the HYDROSIL™ solution (10 wt. %) was added to the HYDROSIL™-loaded POLY-PORE® particles and stirred until homogeneous. The particles were dried again in a 60° C. oven overnight. A free-flowing powder that contained 20% HYDROSIL™, by weight, was obtained.

### Example 9

Forty grams of molten stearyl alcohol and 60 g of molten shea butter were admixed until homogeneous. Ten grams of the loaded microparticles of Example 5 (containing 20% lysine hydrochloride, by weight) were dispersed in 50 g the molten wax mixture at 60° C. with stirring. The resulting molten mixture was sprayed through a two fluid nozzle at an operating pressure of 2 to 5 psi (pounds per square inch) to atomize the mixture into a cold water bath. The resulting solid microparticles were filtered, then dried in a vacuum oven at room temperature. The final loaded microparticles contained 3.3% lysine hydrochloride, 13.4% MICROSPONGE®, 33.3% stearyl alcohol, and 50.0% shea butter, by weight.

### Example 10

Sixty grams of molten stearyl alcohol and 40 g of molten shea butter were admixed until homogeneous. Ten grams of the loaded microparticles of Example 6 (containing 30% lysine hydrochloride, by weight) were dispersed in 70 g the molten wax mixture at 60° C. with stirring. The resulting molten mixture was sprayed through a two fluid nozzle to atomize the mixture at an operating pressure of 2 to 5 psi into a cold water bath. The resulting solid particles were filtered, then dried in a vacuum oven at room temperature. The final loaded microparticles contained 3.75% lysine HCl, 8.75% POLY-PORE®, 52.5% stearyl alcohol, and 35.0% shea butter, by weight.

### Example 11

Example 10 was repeated, except a 1:1 weight mixture of stearyl alcohol and shea butter was used to provide microparticles containing a final composition of 3.75% lysine hydrochloride, 8.75% POLY-PORE® E100, 43.75% stearyl alcohol, and 43.75% shea butter, by weight.

### Example 12

Sixty grams of molten stearyl alcohol and 40 g of molten shea butter were admixed until homogeneous. Ten grams of the loaded microparticles of Example 7 (containing 40% lysine hydrochloride, by weight) were dispersed in 56.6 g the molten wax mixture at 60° C. with stirring. The resulting molten mixture was sprayed through a two fluid nozzle at an operating pressure of 2 to 5 psi to atomize the mixture into a cold water bath. The resulting solid microparticles were filtered, then dried in a vacuum oven at room temperature. The final loaded microparticles contained 6.0% lysine HC1, 9.0% POLY-PORE®, 51.0% stearyl alcohol, and 34.0% shea butter, by weight.

### Example 13

Dow Coming 2503 wax (INCI name: stearyl dimethicone, 50 g) was admixed with 50 g of Dow Coming ST-Wax 30 (INCI name: C30-45 alkyl methicone). The resulting wax mixture was heated to 70° C. to melt, then stirred till until homogeneous. Ten grams of the microparticles obtained in Example 8 (containing 20% HYDROSIL™, by weight) were dispersed in 60 g of the molten wax mixture at 70° C. with stirring. The resulting mixture was sprayed into small droplets through a two fluid nozzle using a steam of inert gas for atomization. A cold water bath was used to collect the particles. The resulting particles were filtered, then dried in a vacuum oven at room temperature. The final product contained 2.86% HYDROSIL™, 11.44% POLY-PORE®, 42.85% DC 2503 wax and 42.85% ST-Wax 30, by weight.

### Example 14

The experiment in Example 13 was repeated, except a mixture of stearyl alcohol and shea butter was used in place of the siloxane wax mixture. The weight ratio of stearyl alcohol to shea butter was 3:2 by weight. The final microparticles contain 2.86% HYDROSIL™, 11.44% POLY-PORE® E 100, 51.42% stearyl alcohol, and 34.28% shea butter, by weight.

### Example 15

### Dihydroxyacetone (DHA) Oil-In-Water Lotion

In some experiments, a DHA oil in water lotion was used as a base into which a 5% DHA was added from a 50% aqueous solution followed by the addition of a loaded microparticle/matrix delivery system containing either POLY-PORE® or POLYTRAP®. The base formulation was:

| | | Ingredients | Wt. % | Batch (g) |
|---|---|---|---|---|
| 1 | A | WATER, DEIONIZED | 58.9 | 883.5 |
| 2 | A | XANTHAN GUM (2% SOLN) | 15.0 | 225.0 |
| 3 | A | NA2EDTA | 0.1 | 1.5 |
| 4 | B | CETEARYL ALCOHOL 70/30 | 3.0 | 45.0 |
| 5 | B | GLYCERYL STEARATE/PEG100 | 1.5 | 22.5 |
| | | STEARATE | | |
| 6 | B | CAPRYLIC/CAPRIC | 12.5 | 187.5 |
| | | TRIGLYCERIDE | | |
| 7 | B | OCTYLDODECANOL | 2.0 | 30.0 |
| 8 | B | STEARETH-21 | 2.5 | 37.5 |
| 9 | B | BEHENYL ALCOHOL (98%) | 2.5 | 37.5 |
| 10 | C | GLYCOLIC ACID (35%) | 1.0 | 15.0 |
| 11 | D | PHENONIP | 1.0 | 15.0 |
| | | TOTAL | 100.0 | 1500.0 |

Manufacturing Process: Admix A ingredients and mix with propeller agitator until uniform. Admix B ingredients and mix with a propeller agitator until uniform. Heat phases A and B, separately, to 75° C. Then, add phase B slowly into phase A, while homogenizing, cool to 40° C., add phase C and D, and mix together.

### Example 16

The loaded microparticles/matrix delivery systems were placed into oil-in-water (o/w) emulsions that contained DHA to test the ability of the microparticles loaded with potentiator to enhance the tanning rate and to minimize adverse esthetics of color formation in the formulation. For example, spray particles (1 g) obtained in Example 9 were placed in 5 g of the DHA oil-in-water lotion described in Example 15, followed by adding 0.67 g of a 50% aqueous DHA solution. A control was made by adding 10 g of a 50% DHA aqueous solution to 90 g of the DHA oil-in-water lotion. A sample with the unloaded amine potentiator was prepared by adding 10 g of a 10% lysine HCl solution to 80 g of the DHA oil in water lotion and 10 g of the 50% DHA aqueous solution. The color development of the tanning composition after the addition of the particles was recorded by an X-Rite colorimeter and photographed. In all cases, the unloaded amine-containing potentiator, or the potentiator loaded onto microparticles coated with a matrix material, was added to the composition to provide a same final concentration of potentiator in the final formulation. All compositions also contained a same amount of DHA. The color of the composition was measured weekly for 12 weeks after adding the potentiator to the composition.

### Example 17

A 5% DHA gel was used as a base in various experiments. The formulation of the DHA gel was:

| | Ingredients | Wt. % | Batch (g) |
|---|---|---|---|
| 1 | WATER, DEIONIZED | 93.0 | 930.0 |
| 2 | DHA | 5.0 | 50.0 |
| 3 | CARBOMER, ULTREZ 10 | 0.3 | 3.0 |
| 4 | NaOH (20%) | 0.7 | 7.0 |
| 5 | PHENONIP | 1.0 | 10.0 |
| | TOTAL | 100.0 | 1000.0 |

Manufacturing process: DHA was dispersed in deionized water, and the resulting dispersion was stirred until homogeneous and transparent. The CARBOMER was added slowly to the DHA solution with vigorous agitation, followed by neutralizing the dispersion with a 20% sodium hydroxide, and finally adding phenonip and mixing until homogeneous.

### Example 18

To 90 g of the 5% DHA gel obtained in Example 17, microparticles obtained in Example 12 (10 g) were added to prepare a gel containing 4.5% DHA and 0.6% lysine hydrochloride. A control sample was prepared by adding 10 g water to 90 g of a 5% DHA gel. A third sample was prepared by adding 10 g of a 6% lysine HCl solution to 90 g of the 5% DHA gel. The unloaded amine potentiator, or the potentiator loaded onto microparticles coated with a matrix material, was added to the composition to provide a same final concentration of potentiator in the final formulation. All compositions also contained a same amount of DHA. The color development of the tanning composition after the addition of the particles was recorded by an X-Rite colorimeter and photographed.

### Example 19

In some experiments, a water-in-oil lotion was used as a base onto which DHA was added from a 50% aqueous DHA solution to provide a final concentration of 5% DHA in the formulation, followed by the addition of spray particles containing either POLY-PORE® or POLYTRAP® loadings. The base formulation was:

| | | Ingredients | Wt. % | Batch (g) |
|---|---|---|---|---|
| 1 | A | Water, deionized | 72.4 | 674.0 |
| 2 | A | Sodium chloride | 0.5 | 5.0 |
| 3 | A | Disodium EDTA | 0.1 | 1.0 |
| 4 | B | Emulsifier (ABIL WE 09) | 3.0 | 30.0 |
| 5 | B | ABIL Wax 9801 | 1.0 | 10.0 |
| 6 | B | Cyclomethicone (DC 345) | 22.0 | 220.0 |
| 7 | C | Germaben II | 1.0 | 10.0 |
| | | TOTAL | 100.0 | 1000.0 |

Manufacturing Process: Combine phase A ingredients, heat to 50° C. to dissolve the ingredients, then cool the mixture to room temperature: Combine phase B ingredients and homogenize at 2000 to 3000 rpm until homogeneous. Add phase A into phase B slowly under homogenizing at 2000 to 3000 rpm, then continue homogenization at 5000 to 6000 rpm for 10 minutes.

### Example 20

To test the ability of loaded microparticles of potentiator to enhance the tanning rate or to minimize adverse esthetics on the formulation, the loaded microparticles were incorporated into a water-in-oil (w/o) composition that contained DHA. In this example, a commercial self-tanning lotion was used. For example, 10 g of spray particles obtained in Example 10 were placed into 65 g of the commercial DHA water-in-oil lotion containing 5% DHA. The final lotion contained 0.5% lysine HCl and 4.33% DHA, by weight. The samples were stored in a 40° C. oven for stability test. The color of the samples was recorded in weekly for 12 weeks by colorimeter and photographs. A photograph of the sample aged for 12 weeks was compared to a control sample, which contains the same amount of DHA, but no lysine hydrochloride, and a second sample, wherein 10 g of a 3.75% lysine hydrochloride solution was directly added to a 65 g DHA lotion, again to give a final emulsion composition containing 4.33% DHA. The sample containing the wax-coated microparticles developed only a light off-white color, wherein the sample containing a same amount of lysine HCl, but free of loaded microparticles, developed a dark brown color after 12 weeks aging at 40° C. The color of the composition after aging the samples at 40° C. are summarized below. The ΔE and Δb* values were calculated with respect to the color measured at time 0, when the samples were freshly made. A higher ΔE value indicates greater change in color of the sample.

| Sample (wt. %) | ΔE | ΔL* | Δa* | Ab* |
|---|---|---|---|---|
| Control (4.33% DHA) | 3.58 | −3.54 | 0.004 | 0.53 |
| 0.5% Lysine HCl | 27.98 | −21.37 | 4.86 | 17.36 |
| 1% POLY-PORE ® E100; | 3.92 | −2.64 | −0.31 | 2.89 |
| 0.5% Lysine HCl; 7.0% | | | | |
| Stearyl Alcohol; 4.7% Shea Butter | | | | |

### Example 21

### Efficacy Measurement

The in vitro efficacy of the sample of Example 20 was measured on VITRO-SKIN® (IMS, Inc),. A 42 mg portion of the lotion was rubbed into 8.4 cm² piece of VITRO-SKIN®. The VITRO-SKIN® was prehydrated in a chamber containing 85% water and 15% glycerin. After applying the lotion, the Vitro-Skin was placed in another chamber containing 20% water and 80% glycerin at 40° C. The color of the in vitro skin was measured for 48 hours. The results are summarized in the following table. Clearly, the in vitro efficacy of the sample is higher than the control. The potentiator enhances the tanning rate and the tanning extent of the DHA lotion.

| | | | | | Control | | | |
|---|---|---|---|---|---|---|---|---|
| | Example 20 | | | | (From Example 20) | | | |
| time (hr) | ΔE | ΔL* | Δa* | Δb* | ΔE* | ΔL* | Δa* | Δb* |
| 0 | 1.96 | −0.24 | −0.45 | 1.90 | 0.83 | −0.05 | 0.24 | −0.80 |
| 2 | 13.82 | −3.50 | 0.80 | 13.34 | 7.37 | −2.25 | 0.29 | 7.01 |
| 4 | 22.64 | −7.29 | 3.23 | 21.19 | 14.74 | −4.12 | 1.69 | 14.06 |
| 6.5 | 28.55 | −10.33 | 5.14 | 26.11 | 20.43 | −7.40 | 2.97 | 18.81 |
| 8 | 30.26 | −10.99 | 5.73 | 27.60 | 22.08 | −7.95 | 3.29 | 20.33 |
| 24 | 39.24 | −16.79 | 8.51 | 34.42 | 32.41 | −12.51 | 5.35 | 29.42 |
| 48 | 39.44 | −17.75 | 9.56 | 33.90 | 32.86 | −12.51 | 5.63 | 29.85 |

### Example 22

Using the formulation base described in Example 19, a control formulation that contained 5% DHA, by weight, was prepared. A second formulation containing 5% DHA plus 20% of POLY-PORE® E100 microparticles loaded with 6% lysine HCl and coated with a mixture of 51% stearyl alcohol and 34% Shea butter, by weight, was prepared. An in vivo test is conducted to measure the color development when applied the tanning composition on human skin. Four 9 cm² areas are marked on the forearm of one subject. The color of the skin was measured using a X-Rite SP 62 color meter. All areas were treated with 38 mg of the formulations. The first two areas were treated with the control formulation and the other two areas were treated with the formulation containing the wax-coated POLY-PORE® E100 polymeric particles loaded with lysine hydrochloride. The color of the skin was recorded as a function of time. Between the end of the first day and the 22-hour time point, the subject washed as normal. The results are listed with respect to the color change (delta E) from the skin before application of the lotions.

| | ΔE POLY-PORE ® | ΔE Control |
|---|---|---|
| time (hr) | Formulation | (5% DHA) |
| 1 | 1.68 | 1.22 |
| 2 | 3.52 | 1.86 |
| 3 | 4.17 | 2.47 |
| 5 | 6.31 | 3.18 |
| 7 | 7.50 | 3.78 |
| 22 | 7.28 | 3.85 |

### Example 23

### Loading Lysine onto POLYTRAP® 6603

A lysine solution was prepared by dissolving 70 g lysine in 100 g DI water. The mixture was stirred until the lysine was completely dissolved. The aqueous (34 g) solution was added to 100 g POLYTRAP® 6603 microparticles in droplets while stirring. After mixing until homogenous, the microparticles loaded with lysine were placed in a 60° C. vacuum oven overnight to remove the water. A free-flowing powder that contained 12.3% lysine, by weight, was obtained.

### Example 24

Shea butter (100 g) was melted, then loaded onto 50 g of the loaded microparticles of Example 23, which were preheated to 50° C. The microparticles were stirred until homogenous. The final weight percentages of shea butter and lysine in the loaded microparticles was 66.7% and 4.1% respectively.

### Example 25

In this example, a commercial self-tanning water-in-oil lotion was used. The shea butter-coated POLYTRAP® microparticles loaded with lysine obtained in previous Example 22 (9 g) were placed in 91 g of a commercial water-in-oil lotion containing 4% DHA under stirring until homogenous. The final lotion contained 0.37% lysine and 3.64% DHA, by weight. The samples were placed in a 40° C. oven for a stability test. The color of the samples was recorded in a weekly base. A yellow color developed after overnight storage, and a dark brown color developed after only 4 weeks at 40° C.

In accordance with an important feature of the present invention, the active compound can be any of a wide variety of compounds, either water soluble or oil soluble. Often, the active compound is a topically-active compound. A composition containing a present delivery system, therefore, can be applied to the skin, and the active compound then performs its intended function.

Although the previous discussion is directed primarily to self-tanning compounds, the active compound can be a different type of compound, such as a fragrance, a pesticide, or similar types of active compounds, like drugs and therapeutic agents.

The active compound often is a water-soluble or water-dispersible compound, i.e., is hydrophilic. However, the active compound can be oil soluble or oil dispersible, i.e., is hydrophobic. In other embodiments, the active compound is a mixture of compounds, either all hydrophilic, all oleophilic, or a mixture of hydrophilic and oleophilic compounds.

The topically-active compound, therefore, can be one of, or a mixture of, a cosmetic compound, a medicinal-active compound, or any other compound that is useful upon topical application to the skin or hair. Such topically-active compounds include, but are not limited to, hair-growth promoters, deodorants, skin-care compounds, antioxidants, hair dyes, antibacterial compounds, antifungal compounds, anti-inflammatory compounds, topical anesthetics, sunscreens, and other cosmetic and medicinal topically-effective compounds.

For example, a skin conditioner can be the active compound of a composition of the present invention. Skin conditioners include, but are not limited to, humectants, such a fructose, glucose, glycerin, propylene glycol, glycereth-26, mannitol, and urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, almond oil, grape seed oil, and castor oil, and mixtures thereof. Numerous other skin conditioners are listed in the CTFA Cosmetic Ingredient Handbook, Tenth Ed., T. E. Gottshalck, et al, ed., The Cosmetic, Toiletry and Fragrance Association (2004), (hereafter CTFA Handbook), pages 2392-2395, incorporated herein by reference.

In addition, the topically-active compound can be a hair dye, such as, but not limited to, m-aminophenol hydrochloride, p-aminophenol sulfate, 2,3-diaminophenol hydrochloride, 1,5-naphthalenediol, p-phenylenedi amine hydrochloride, sodium picramate, cationic dyes, anionic dyes, FD&C dyes, like Blue No. 1, Blue No. 2, Red No. 3, Red No. 4, or Red No. 40, D&C dyes, like Yellow No. 10, Red No. 22, or Red No. 28, and pyrogallol. Numerous other hair dyes are listed in the CTFA Handbook, pages 2351-2354, incorporated herein by reference.

The topically-active compound also can be an antioxidant, like ascorbic acid or erythorbic acid, or a fluorescent whitening agent or optical brightener, like a distyrylbiphenyl derivative, stilbene or a stilbene derivative, a pyralozine derivative, or a coumarin derivative. In addition, a hair growth promoter can be the topically-active compound.

The topically-active compound also can be a deodorant or antiperspirant compound, such as an astringent salt or a bioactive compound. The astringent salts include organic and inorganic salts of aluminum, zirconium, zinc, and mixtures thereof. The anion of the astringent salt can be, for example, sulfate, chloride, chlorohydroxide, alum, formate, lactate, benzyl sulfonate, or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides having the general formula Al₂(OH)ₓ_{y}•XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts also referred to as zirconyl salts and zirconyl hydroxy salts, and represented by the general empirical formula ZrO(OH)_{2-nz}L_{z}, wherein z varies from about 0.9 to about 2 and is not necessarily an integer; n is the valence of L; 2-nz is greater than or equal to 0; and L is selected from the group consisting of halides, nitrate, sulfamate, sulfate, and mixtures thereof.

Exemplary deodorant compounds, therefore, include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohythex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichlorohydrex PG, aluminum chloride, aluminum zirconium pentachlorohydrate, chlorophyllin copper complex, numerous other useful antiperspirant compounds listed in the CTFA Handbook at page 2329-2330, incorporated herein by reference, and mixtures thereof. The active compound also can be a fragrance that acts as a deodorizer by masking malodors. Numerous fragrance compounds are listed in the CTFA Handbook, pages 2345-2346, incorporated herein by reference.

In addition, other compounds can be included as the topically-active compound in an amount sufficient to perform their intended function. For example, if the composition is intended to be a sunscreen, then compounds such as benzophenone-3, trihydroxycinnamic acid and salts, tannic acid, uric acids, quinine salts, dihydroxy naphtholic acid, an anthranilate, diethanolamine methoxycinnamate, p-aminobenzoic acid, phenylbenzimidazole sulfonic acid, PEG-25, p-aminobenzoic acid, or triethanolamine salicylate can be used as the active compound.

Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the active compound. Other sunscreen compounds are listed in CTFA Handbook, pages 2397-2399, incorporated herein by reference.

Similarly, topically-active compounds, like antifungal compounds, antibacterial compounds, anti-inflammatory compounds, topical anesthetics, skin rash, skin disease, and dermatitis medications, and anti-itch and irritation-reducing compounds can be used as the active compound in the compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidone-iodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin, clindamycin phosphate, 5,7-dichloro-8-hydroxyquinoline, and the like; anti-inflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin bleaching agents, skin protestant, such as allantoin, and antiacne agents, such as salicylic acid, also can be incorporated in a composition of the present invention in an amount sufficient to perform its intended function. Other topically active compounds are listed in Remington's Pharmaceutical Sciences, 17th Ed., Merck Publishing Co., Easton, Pa. (1985), pages 773-791 and pages 1054-1058 (hereinafter Remington's), incorporated herein by reference.

In the preparation of a delivery system of the present invention, the active compound first is loaded onto the microparticles, then the matrix material is applied to the loaded microparticles.

The active compound also can be an oral care compound. A variety of oral care compounds can be incorporated into the polymeric microparticles. The oral care compounds include, but are not limited to:

(a) antibacterials, such as a halogenated diphenyl ethers, e.g., 2′,4,4′-trichloro-2-hydroxy-diphenyl ether, known under the trade name triclosan, and 2,2′-dihydroxy-5,5′-dibromo-diphenyl ether; 2,2′-methylenebis-4-4-chloro-6-bromo-phenol); halogenated salicylanilides; halogenated carbanilides; sodium tripolyphosphate; cetyl pyridinium chloride; benzalkonium chloride; sodium hypochlorite; hexachlorophene; thymol; cresols; guaiacol; eugenol; creosote; copper sulphate; copper-(ethyl) maltol; zinc- and stannous salts, such as zinc citrate and sodium zinc citrate; stannous pyrophosphate; and sanguinarine extract;

(b) caries prophylactics, such as a fluoride ion source like sodium fluoride, stannous fluoride, and sodium monofluorophosphate; sodium chloride; and sodium bicarbonate;

(c) a tooth whitener, such as hydrogen peroxide, sodium percarbonate, sodium perborate, po-tassium peroxydiphosphate, and organic peracids;

(d) an antiplaque agent, such as a silicone polymer;

(e) an analgesic, such as codeine, aspirin, acetaminophen, propoxyphene, meperidine, and benzocaine;

(f) flavors, such as spearmint oil, methyl salicylate, cinnamon oil, peppermint oil, clove oil, saccharin, thymol, menthol, and eucalyptus; and

(g) surfactants, such as sodium lauryl sulfate.

The compositions of the present invention also can include optional ingredients traditionally included in cosmetic, medicinal, and other such compositions. These optional ingredients include, but are not limited to, dyes, fragrances, preservatives, antioxidants, detackifying agents, and similar types of compounds. The optional ingredients are included in the composition in an amount sufficient to perform their intended function.

Obviously, many modifications and variations of the invention as hereinbefore set forth can be made without departing from the spirit and scope thereof and, therefore, only such limitations should be imposed as are indicated by the appended claims.

1. A delivery system comprising:
(a) an adsorbent polymer microparticle;
(b) an active compound, said active compound adsorbed onto said adsorbent polymer microparticle to provide a loaded microparticle; and
(c) a matrix material, said matrix material encasing the loaded microparticle, and present in an amount of at least 68% to about 99%, by total weight of the delivery system.

2. (canceled)

3. (canceled)

4. The delivery system of claim 1 wherein the active compound is water soluble and the matrix material is oil soluble and a solid at 25° C., and is selected from the group consisting of a wax, a synthetic wax, a fatty alcohol, an ethoxylated fatty alcohol, a C8-C20 fatty acid, a hydrocarbon, a fat, an oil, a silicone oil, a silicone wax, a water-insoluble ester, and mixtures thereof.

5. (canceled)

6. The delivery system of claim 1 wherein the active agent is oil soluble and the matrix material is water soluble and is selected from the group consisting of a poly(acid), a polyol, an alkanolamide, a water-soluble polymer, a biological polymer, a gum, a carbohydrate, a cellulose derivative, a sorbitan derivative, and mixtures thereof.

7. (canceled)

8. The delivery system of claim 1 wherein the active compound is selected from the group consisting of a topically-active compound, an oral care compound, a fragrance, a pesticide, a drug, and a therapeutic agent.

9. The delivery system of claim 8 wherein the topically-active compound is selected from the group consisting of a hair-growth promoter, a deodorant, an antiperspirant compound, a skin-care compound, an antioxidant, a hair dye, a self-tanning compound, an antibacterial compound, an antifungal compound, an anti-inflammatory compound, a topical anesthetic, a sunscreen, a dermititis or skin disease medication, and mixtures thereof.

10. (canceled)

11. The delivery system of claim 1 wherein the active compound is selected from the group consisting of a silicone, isopropyl myristate, vitamin E acetate, ascorbic acid, retinoid, salicylic acid, benzoyl peroxide, zinc pyrithione, benzophenone-3, benzyl acetate, a fragrance, glycolic acid, and mixtures thereof.

12. (canceled)

13. (canceled)

14. (canceled)

15. (canceled)

16. (canceled)

17. (canceled)

18. A composition comprising
(a) a first active compound; and
(b) a delivery system comprising:
(i) a second active compound loaded onto polymeric microparticles; and
(ii) a matrix material encasing the loaded polymeric microparticles of (i), wherein the matrix material is present at greater than 68% to about 99%, by total weight of the matrix material and the loaded polymeric microparticles.

19. The composition of claim 18 wherein:
(a) the first active compound comprises about 0.1% to about 10%, by weight, of a self-tanning compound; and
(b) the second active compound comprises about 2% to about 80% of a self-tanning potentiator loaded onto polymeric microparticles.

20. (canceled)

21. The composition of claim 19 wherein the self-tanning compound comprises dihydroxyacetone, L-erythrulose, or a mixture thereof.

22. (canceled)

23. (canceled)

24. The composition of claim 19 wherein the self-tanning potentiator comprises an amino acid, an amino acid salt, a diamine, a triamine, an amino-containing polymer, or a mixture thereof.

25. The composition of claim 24 wherein the self-tanning potentiator comprises lysine, glycine, arginine, or their salts, amodimethicone, methoxy amodimethicone/silesquioxane copolymer, a linear polyethylenimine, a branched polyethylenimine, a dendritic amino polymer, poly(lysine), poly(argine), or mixtures thereof.

26. (canceled)

27. (canceled)

28. (canceled)

29. (canceled)

30. The composition of claim 18 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

31. (canceled)

32. (canceled)

33. (canceled)

34. The composition of claim 18 wherein the matrix material is solid at 25° C. and is selected from the group consisting of a C8-C20 alcohol, a fatty alcohol ethoxylated with one to three moles of ethylene oxide, a C8-C20 fatty acid, a hydrocarbon wax, an oil, an ester containing at least 10 carbon atoms, a butter, and mixtures thereof.

35. (canceled)

36. (canceled)

37. (canceled)

38. (canceled)

39. The composition of claim 18 wherein the composition is a water-in-oil emulsion, an oil-in-water emulsion, a water-in-silicone emulsion, an aqueous gel, or a nonaqueous gel.

40. (canceled)

41. (canceled)

42. (canceled)

43. (canceled)

44. A process for producing a delivery system of claim 1 comprising applying the matrix material to the loaded polymeric microparticles via a congealing process.
